(19) United States
(12) Patent Application Publication
(10) Pub. No.: US 2023/0181373 A1
(43) Pub. Date: Jun. 15, 2023

(54) ADHESIVE BANDAGE SYSTEM FOR MEDICATION DELIVERY AND INDICATION OF PROCEDURE LOCATION

(71) Applicant: Stephen Welsh, Hurricane, UT (US)

(72) Inventor: Stephen Welsh, Hurricane, UT (US)

(21) Appl. No.: 18/065,214

(22) Filed: Dec. 13, 2022

Related U.S. Application Data
(60) Provisional application No. 63/288,852, filed on Dec. 13, 2021.

(51) Int. Cl. A61F 13/00; A61F 13/02; A61M 5/42

(52) U.S. Cl. CPC A61F 13/00063; A61F 13/0266; A61M 5/427; A61F 2013/00285

(57) ABSTRACT

An adhesive bandage system features a first member with an aperture substantially circumscribed by an adhesive layer couplable to a patient's skin. A second member of the adhesive bandage is removably couplable to the first member, having an adhesive along an outer perimeter on a first side, the outer perimeter sized so as to overlap with the first member. In some methods of use, a user may adhere the first member to a patient skin, administer a topical medication through the aperture, and then adhere the second member so as to cover the aperture. The second member may be removed, as needed, to either add medication, replace the medication with a new medication, or to have access to an injection site with a needle or catheter.

### CROSS-REFERENCE TO RELATED APPLICATIONS

This application claims the benefit of U.S. Provisional Application Ser. No. 63/288,852, filed on Dec. 13, 2021, which is incorporated herein by reference.

### TECHNICAL FIELD

The present disclosure relates to bandages. More particularly, the present disclosure relates to an adhesive bandage having a removable member or portion for medication delivery and situs indication.

### BACKGROUND

Topical medications are used in a variety of settings and with a variety of patients. The most common type of topical medication is simply an ointment or cream that is applied directly to the skin. While this can be an effective delivery method, it is often messy, as the ointment or cream may come into contact with a patient's clothes or other surfaces. In some instances, medication (e.g., testosterone) may also be inadvertently transferred to someone else, like a spouse or child, which may have serious consequences. Often times, the ointment or cream can be mistakenly rubbed off, decreasing the effectiveness of the medication. Additionally, it is difficult to produce a metered dose when simply applying directly to the skin without coverings.

To solve this issue, some topical medications are delivered using transdermal patches. However, these patches often underperform in relation to ointments and creams, and require a medical practitioner to stock a variety of patches for use, which is costly. Further, the amount or dosage of the patch is not variable, which leads to either difficulties in practitioners administering proper doses or results in the practitioner stocking additional patches with varying dosages of each type.

Additionally, it may be beneficial for some ointments or creams to be removed after a short time so that other medications may be administered. For example, a topical anesthetic may be useful for numbing an injection site prior to an injection. Or a topical analgesic may be beneficial post injection. However, these ointments and creams must be applied some time in advance of the injection to be effective. As described earlier, a patient applying a cream or ointment beforehand may not last, as it will get rubbed off before arriving. If applied by the medical practitioner, the patient then occupies time and space while waiting for the topical ointment to take effect prior to the shot. As a result, ointments and creams are not utilized as often as they could be. Further, even if the ointment is used, once it is cleared from the skin, it may be difficult to identify the numb location, resulting in injections outside the numb location.

Accordingly, there is a need for a bandage for use with creams, ointments and solutions, that allows metered doses, and that provides an indication to a user of the location of the applied medication. The present disclosure seeks to solve these and other problems.

### SUMMARY OF EXAMPLE EMBODIMENTS

In some embodiments, an adhesive bandage system comprises a first member and a second member, the second member removably replaceable within an aperture of the first member. The first member comprises an adhesive on a first side for adhering to the skin of a patient with an aperture in the center thereof. In some embodiments, the second member comprises an adhesive along its outer perimeter on a first side, the outer perimeter sized so as to overlap with the first member.

In some methods of use, a user may adhere the first member to a patient. The user may then place the desired medication in the aperture in an amount and dosage appropriate to the patient and treatment, the medication being in direct contact with the skin. The second member may then be placed so as to cover the aperture, the edges of the second member contacting the first member to removably adhere the second member to the first member. The second member may be removed to either add medication, replace the medication with a new medication, or to have access to an injection site.

In some embodiments, the second member may be preassembled to the first member. A user may then deposit the medication, on a bottom side, into the aperture that is covered by the second member. The first member may then be adhered to a patient. When desirable, a user may remove the second member to expose the skin. If additional medication is deposited, a second member may then be replaced on the first member.

In some embodiments, a medication may be sealed in the aperture by the manufacturer. A user may then remove a release liner, exposing an adhesive layer of the first member and also exposing the medication in the aperture for contact with, and absorption across, the skin.

In some embodiments, rather than placing an ointment or cream within the aperture, the second member may be a patch comprising medication.

### BRIEF DESCRIPTION OF THE DRAWINGS

FIG. 1 illustrates a top perspective view of an adhesive bandage system;

FIG. 2 illustrates a bottom perspective view of an adhesive bandage system;

FIG. 3 illustrates a side perspective view of an adhesive bandage system;

FIG. 4 illustrates a top perspective view of an adhesive bandage system adhered to a patient;

FIG. 5 illustrates a second member of an adhesive bandage system being removed from a first member;

FIG. 6 illustrates a first member of an adhesive bandage system adhered to a patient with an exposed aperture;

FIG. 7 illustrates a bottom perspective view of an adhesive bandage system receiving medication in an aperture;

FIG. 8 illustrates a first member of an adhesive bandage system adhered to a patient with medication being deposited on the skin through an aperture;

FIG. 9 illustrates a rear, disassembled view of an adhesive bandage system;

FIG. 10 illustrates a side, front perspective view of an adhesive bandage system with a port;

FIG. 11 illustrates a rear, disassembled view of an adhesive bandage system comprising a transdermal patch;

FIG. 12 illustrates a side, disassembled view of an adhesive bandage system;

FIG. 13 illustrates a front, side perspective view of an adhesive bandage system on a user;

FIG. 14 illustrates a front, side perspective view of an adhesive bandage system on a user receiving an injection;

FIG. 15 illustrates a side, disassembled view of an adhesive bandage system; and

FIG. 16A illustrates a side, disassembled view of an adhesive bandage system;

FIG. 16B illustrates a side, detailed view of an adhesive bandage system; and

FIG. 16C illustrates a side, detailed view of an adhesive bandage system.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As discussed earlier, there is a need for a bandage for use with creams, ointments, and solutions, that allows metered doses, and that provides an indication to a user of the location of the applied medication. The bandage disclosed herein seeks to solve these and other problems.

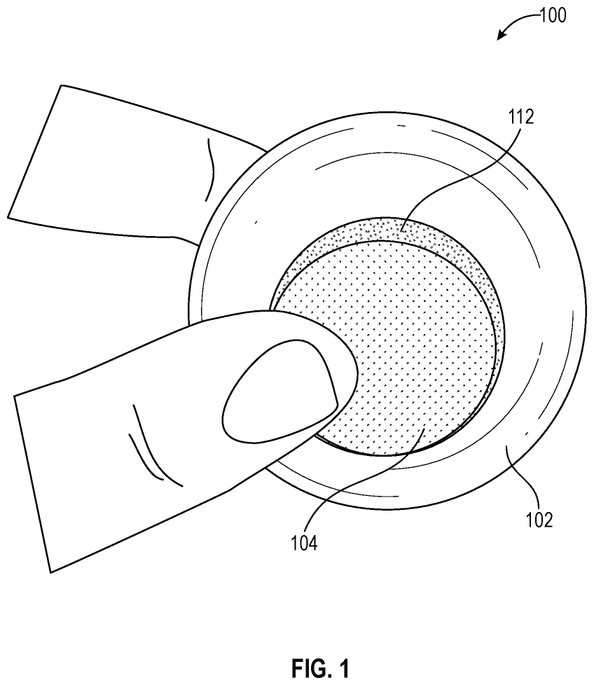

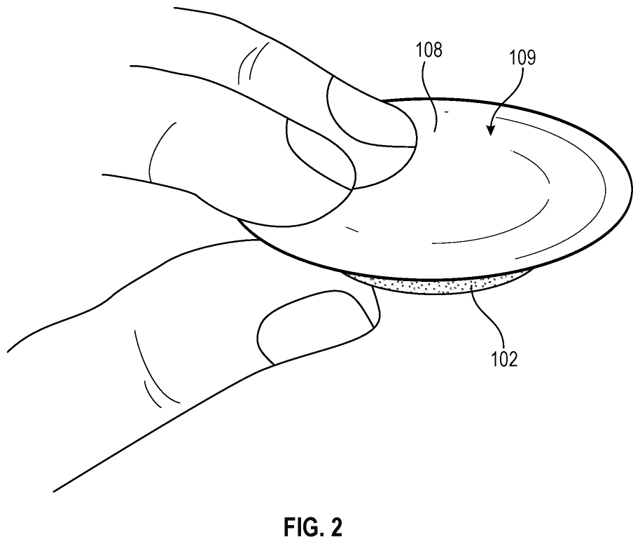

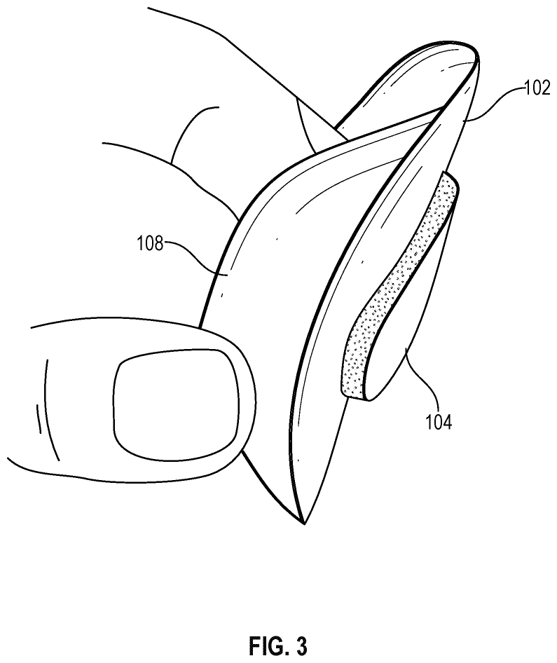

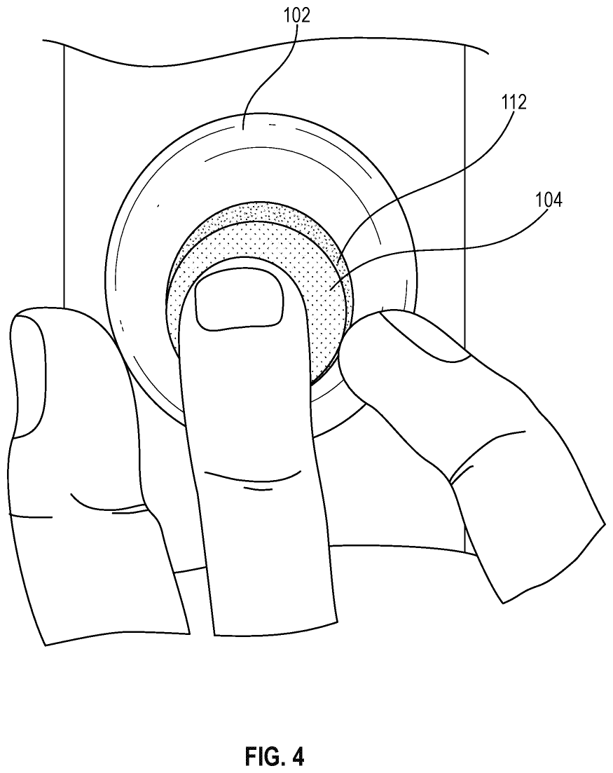

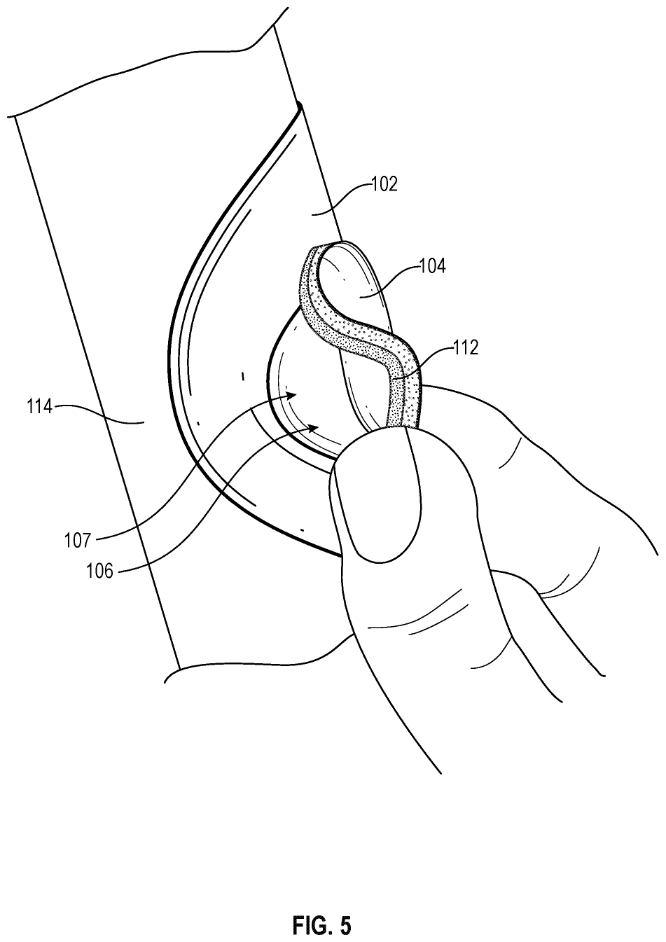

Referring to FIGS. 1-7, in some embodiments, an adhesive bandage system 100 comprises a first member 102, a second member 104, and an aperture 106 (FIG. 5) in the first member 102. The second member 104 is configured to cover and/or be at least partially receivable within the aperture 106. In some embodiments, the first member 102 comprises a release liner 108, such as a plastic or paper film, on a first side 109, that may form a protective layer over an adhesive on the first side 109 (underside) of the first member 102 for adhering to the skin of a patient. The release liner may be substantially coextensive with the adhesive layer. As shown in FIG. 2, the release liner 108 may cover the entire bottom of the first side 109, including the aperture 106.

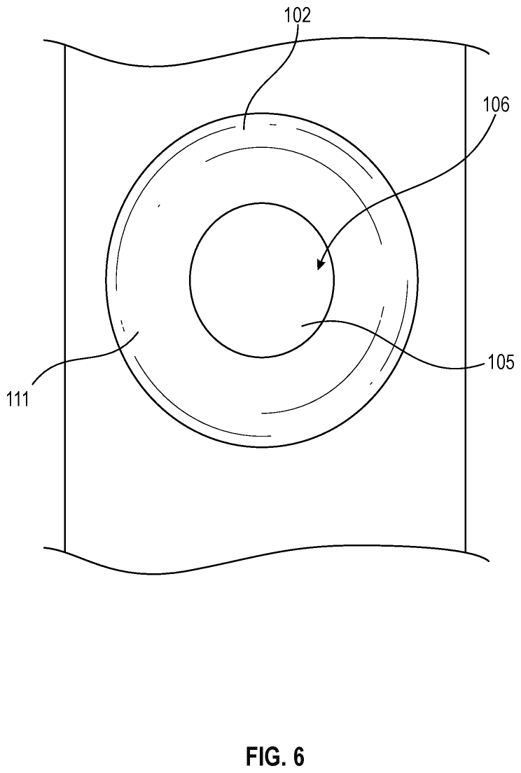

A medication (e.g., lidocaine) may be deposited in the aperture 106 in several ways for contacting the patient's skin. For example, as shown in FIGS. 1-6, in some embodiments, a manufacturer may place a desired medication or substance in the aperture 106 in advance, with the medication/substance remaining sealed between the release liner 108 and the second member 104 until use. In other words, a medication reservoir 107 (FIG. 5) is formed between the release liner 108 and the second member 104. To apply the adhesive bandage system 100 to a patient, a user peels the release liner 108 from the first side 109, thereby exposing an adhesive layer on the first side 109 and also exposing the aperture 106, thereby allowing the medication/substance in the medication reservoir 107 to contact a patient's skin 105. After a predetermined timeframe (e.g., sufficient to numb the skin), the user may remove, as shown in FIG. 5, the second member 104 from the first member 102, thereby exposing the aperture 106 from a top, second side 111 of the first member 106, as best seen in FIG. 6. The user may then clean the skin 105 through the aperture 106 in preparation for the injection or other procedure. Because the first member 102 remains adhered to the skin, the user is able to easily determine the area for the injection by injecting through the aperture 106 (the “bullseye”).

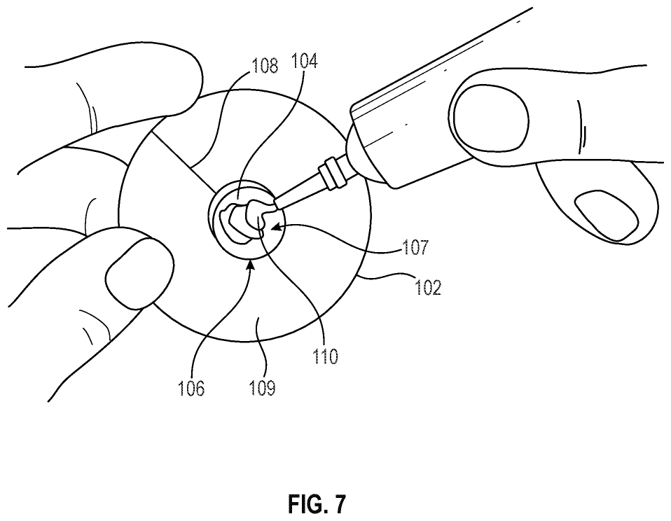

In some embodiments, rather than the manufacturer placing medication or substances in the medication reservoir 107, the medication reservoir 107 may be accessible to a user, as shown in FIG. 7, to add the desired medication 110, with the second member 104 adhered to the first member 102 to contain the medication 110 in the formed medicine reservoir 107. In other words, the release liner 108 in this embodiment does not cover the aperture 106, leaving it accessible to a user. Once the medication 110 is deposited, a user will remove the release liner 108 from the first side 109 and will adhere the first side 109 of the first member 102 to a patient. When a sufficient time has elapsed for the medication to be absorbed, a user may then remove the second member 104, thereby exposing the aperture 106 and the medicine 110 therein. A user may then clean out the medicine and administer the injection or perform a procedure through the aperture 106, which forms the “bullseye” for the user.

It will be appreciated that any suitable substances may be added to the aperture 106 or reservoir 107, such as antiseptics, antifungal agents, antibacterial agents, antiviral agents, hormones, antihistamines, steroids, or any other substance desirable for transdermal application.

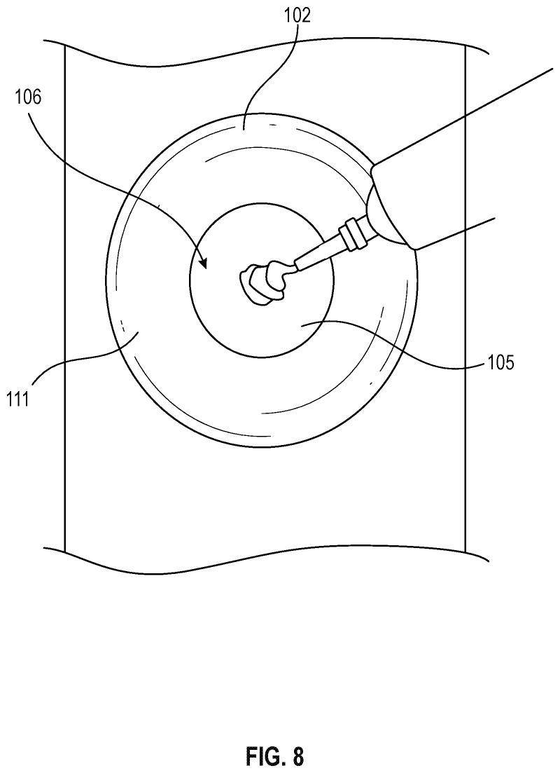

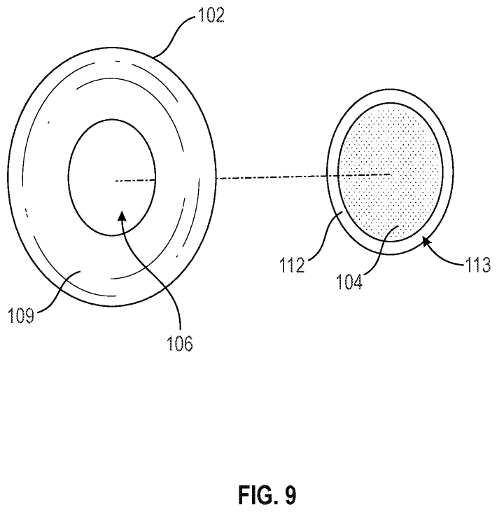

In some embodiments, as shown in FIGS. 8-9, a user may remove a release liner 108 from a first side 109 of the first member 102, adhere the first member 102 on a patient, deposit the medication 110 through the aperture 106 directly onto the skin 105, then seal the aperture 106 using the second member 104, thereby enclosing the medication 110 within the aperture 106 and on the skin 105 of a patient. Accordingly, in some embodiments, as shown in FIG. 9, the second member 104 comprises an adhesive along its outer perimeter 112 on a first side 113, the diameter of the outer perimeter 112 greater than the diameter of the aperture 106, thereby allowing the outer perimeter 112 to adhere to the second side 111 of the first member 102, thereby enclosing the medication 110 in the aperture 106, preventing the medication 110 from inadvertent or premature removal. The second member 104 may be removed to either add medication 110, replace the medication 110 with a new medication 110, or to have access to an injection site for inserting a needle or other medical procedure.

A user may remove the second member 104 to expose the aperture 106 and the skin 105 of the patient. Because the first member 102 remains adhered to the skin, a user or medical provider is able to easily determine where the medication has been in contact with the skin and, therefore, the injection site. For example, for an injection, an anesthetic may be placed in the medication reservoir 107 of the first member 102. Because the reservoir 107 remains covered by the second member 104, the medication is not rubbed off or otherwise prematurely removed from the desired site. When the patient is ready for the injection, the second member 104 may be removed (e.g., peeling it off from the first member 102), exposing the skin and residual medication 110. The aperture 106 allows the skin to be cleaned and sterilized. However, because the first member 102 remains adhered to the subject, the medical provider may confidently inject into the numbed and cleansed skin, the aperture 106 forming a target or bullseye for the injection. Unlike the prior art, which requires injections through layers of adhesive or other film, the adhesive bandage system 100 allows a medical provider to confidently inject directly into the prepared skin of the patient.

Additionally, after an injection, other medications 110 may be inserted into the aperture 106 if desired, such as topical analgesics and/or antibiotics. If additional medication 110 is deposited, a new, non-contaminated, second member 104 may then be replaced on the first member 102, once again sealing the aperture 106 and the medication therein, which forms the medicine reservoir 107.

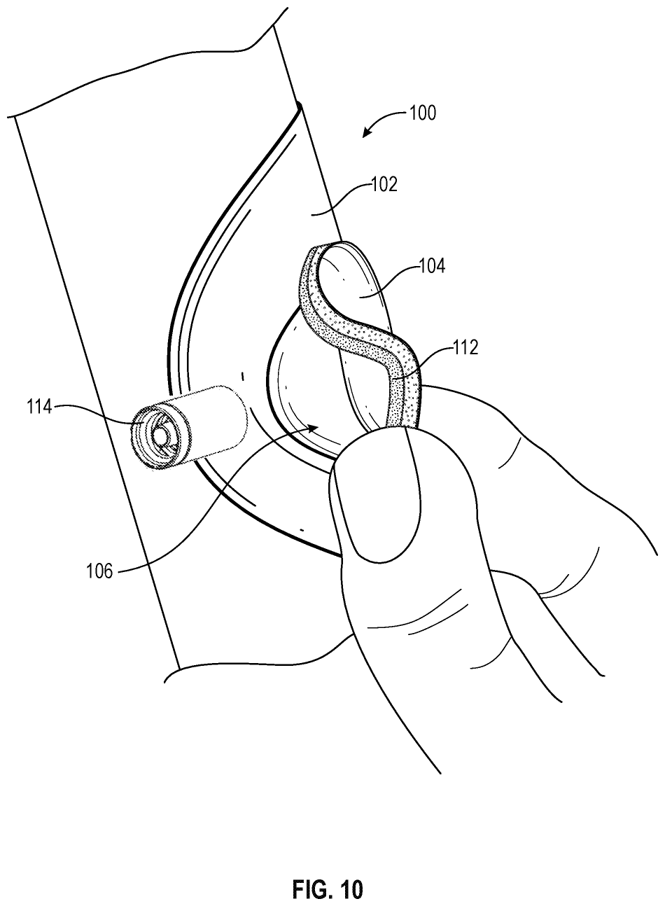

As shown in FIG. 10, in some embodiments, the adhesive bandage system 100 may comprise one or more ports or couplers 114 to connect to, or otherwise secure, other items, such as an IV catheter, tube, needle, etc., to the adhesive bandage system 100. The one or more couplers 114 may be positioned on the first member 102 so as to remain secured to a patient when the second member 104 is removed therefrom. In some embodiments, however, the second member 104 may be configured to receive tubes, needles, catheters, or other accessories as well. The one or more couplers 114 may be configured to anchor and secure catheters to prevent movement and dislodgment of the needle. The one or more couplers 114 may be secured by additional medical dressings and adhesives.

It will be appreciated that the size and shape of the adhesive bandage system 100 may vary for specific uses. Additionally, the first member 102 and/or the second member 104 of the adhesive bandage system 100 may comprise an occlusive or non-occlusive dressing, depending upon the application and use with different topical agents. The occlusive dressing may be an air- and water-tight trauma medical dressing made with a waxy coating as to provide a total seal around the patient's skin. The occlusive dressing may also comprise films, foams, hydrogels, or hydrocolloids. In some embodiments, the non-occlusive dressing may be gauze having natural or synthetic fibers such as cotton, rayon, or polyester. Any topical medication 110 may be used, including, but not limited to, lidocaine, benzocaine, testosterone, estrogen, estradiol, ketoprofen, indomethacin, diclofenac, capsaicin, methyl salicylate, topical antibiotics, gabapentin, or topical corticosteroids.

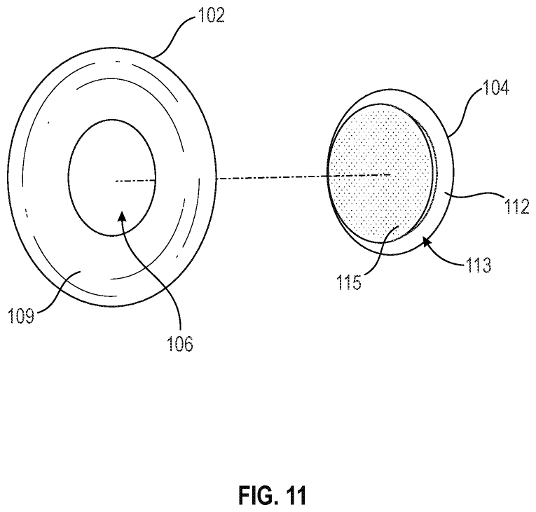

In some embodiments, as shown in FIG. 11, the second member 104 comprises a transdermal patch 115. In other words, rather than depositing medication into the aperture 106 and medicine reservoir 107, the transdermal patch 115 of the second member 104 is received through the aperture 106 where it contacts the skin of a patient. The outer perimeter 112 adheres to the second side of the first member 102, ensuring the transdermal patch 115 remains in contact with the skin. After a predetermined amount of time has elapsed, the second member 104 may be removed by peeling it from the first member 102, the skin may then be cleaned, and the procedure administered through the aperture 106, the aperture 106 functioning as a bullseye.

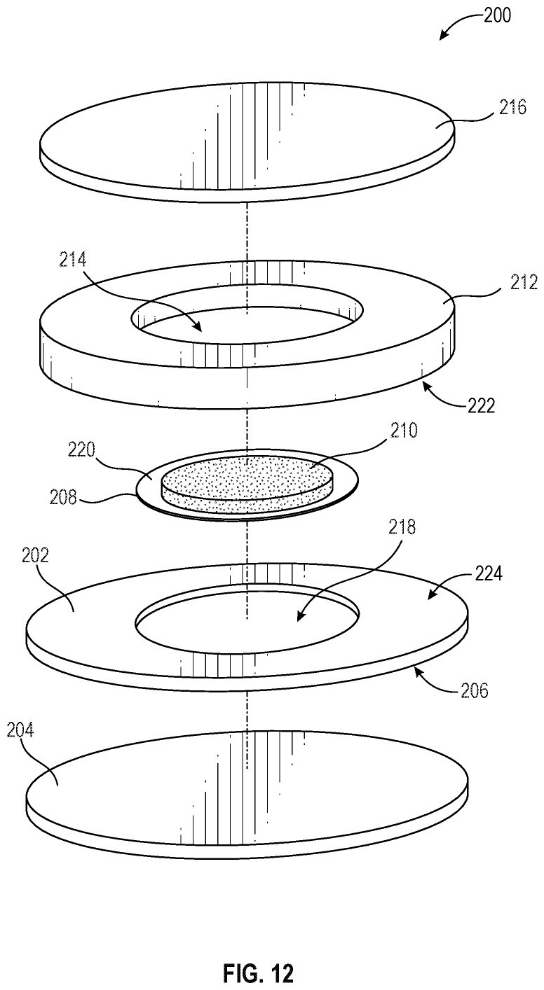

Referring to FIG. 12, in some embodiments, an adhesive bandage system 200 comprises a first member 202, a release liner 204 (e.g., backing paper) coupled to a first side 206 of the first member 202, and a second member 208 comprising medication or other topical ointment/cream 210. The second member 208 may comprise gauze or other non-stick material that allows for the medication to pass therethrough. A third member 212 may protect a pre-packaged medication 210. For example, the third member 212 may comprise an aperture 214 in the center thereof and have a height greater than the height of the medication 210 on the second member 208, thereby allowing the medication to reside within the aperture 214 during packaging and storage, reducing or preventing the medication 210 from coming into contact with a cover 216. The third member 212 may comprise foam or other materials, allowing it to flex, allowing for the bandage to be used on different curves of a patient's body. The third member 212 is manufactured from non-absorbent materials so that the medication is not absorbed thereinto. Lastly, a film or cover 216 encloses and protects the medication 210.

The second member 208 is received within a first aperture 218 of the first member 202. Because the second member 208 is made from gauze, mesh, or other material allowing the medication 210 to pass or seep therethrough, the medication 210 will contact a patient once the release liner 204 is removed. The second member 208 is adhered, around an outer edge 220, to the third member 212. The underside 222 of third member 212 is releasably adhered to top side 224 of the first member 202, allowing a user to remove the third member 212 by peeling it from the first member 202.

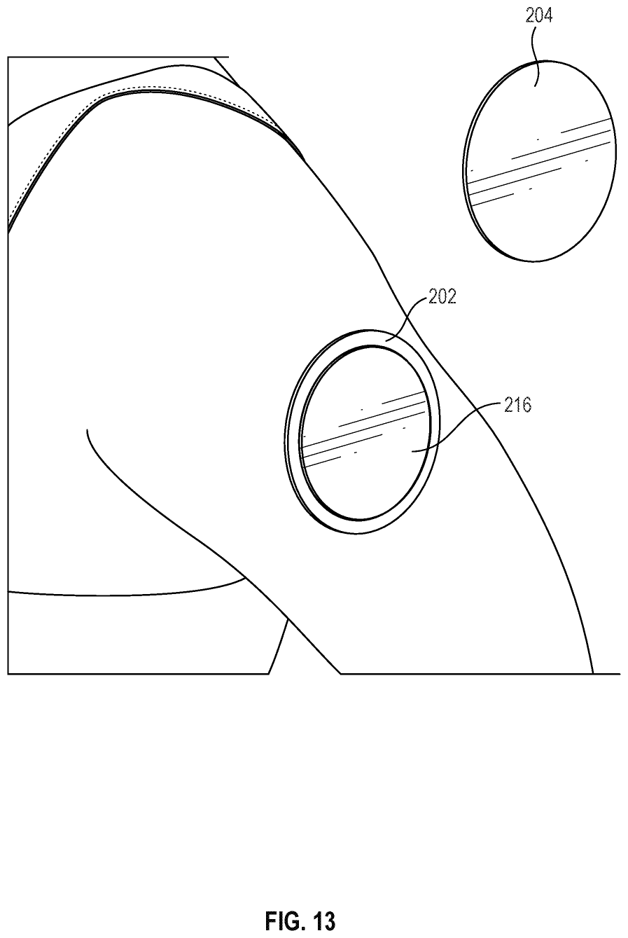

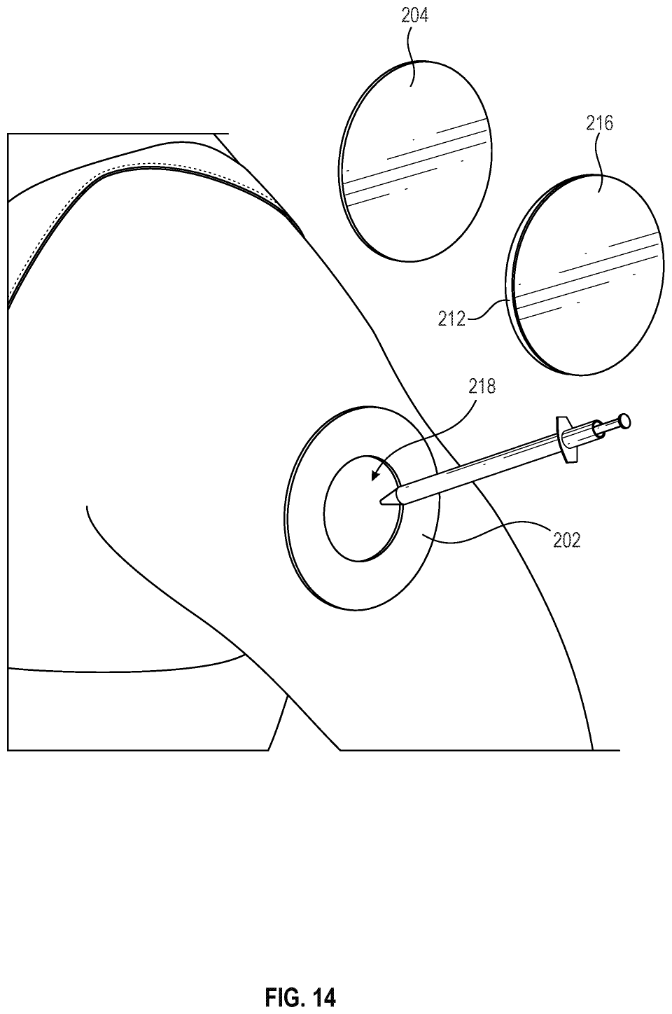

Accordingly, in some methods of use, a user will peel the release liner 204 from the first side 206 of the first member 202, exposing the adhesive on the first side 206. The user will then press the first side 206 onto the desired location on a patient, as shown in FIG. 13, adhering it to the patient. The medication 210 on the second member 208 then contacts and is absorbed into the skin. The medication 210 remains covered and protected by the third member 212 and the cover 216, preventing unwanted removal, contact, or contamination of the medication 210. Once an appropriate amount of time has elapsed for the medication 210 to have its desired effect (e.g., in the case of lidocaine, numbing the skin for an injection), a user may grasp and peel the third member 212 from the first member 202. Because the second member 208 is adhered to the third member 212, it likewise, is removed, along with the cover 216. As a result, the only remaining portion on the patient is the first member 202. A user may then cleanse the skin through the aperture 218 in preparation for the procedure. As shown in FIG. 14, the aperture 218 functions as a target or bullseye, allowing a user (e.g., physician) to easily administer an injection into the prepared area without guessing. While a medication 210 was discussed as being received on the second member 208, it will be appreciated that the second member may also be a transdermal patch with medication disposed therein.

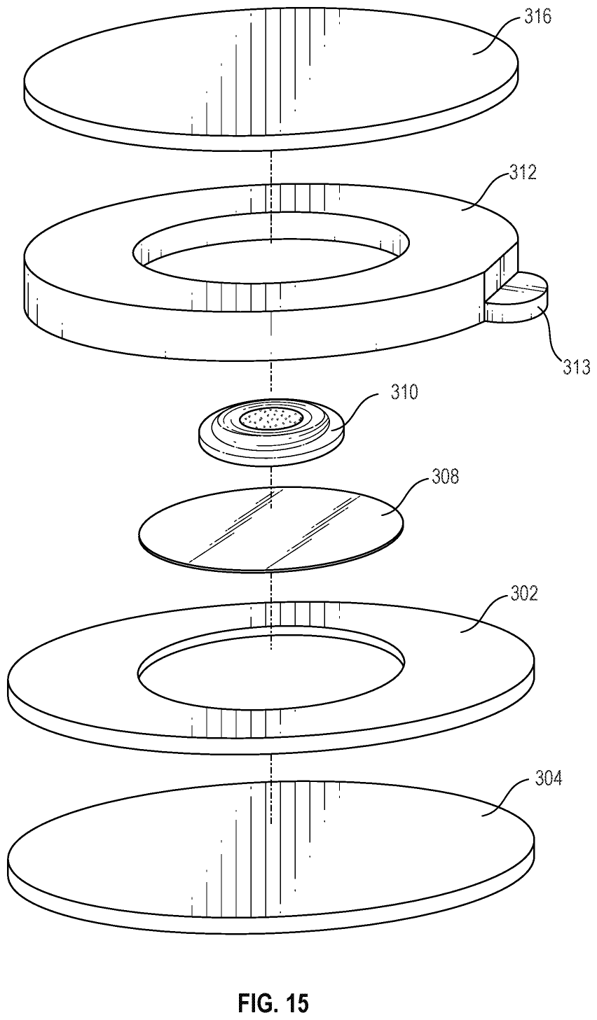

In some embodiments, as shown in FIG. 15, the third member 312 comprises a tab 313 that protrudes beyond the perimeter of the first member 302, allowing a user to easily grasp the tab 313 to peel the third member 312 from the first member 302. As in the prior embodiment, the adhesive bandage may comprise a release liner 304, a second member 308, a medication 310, and a cover 316.

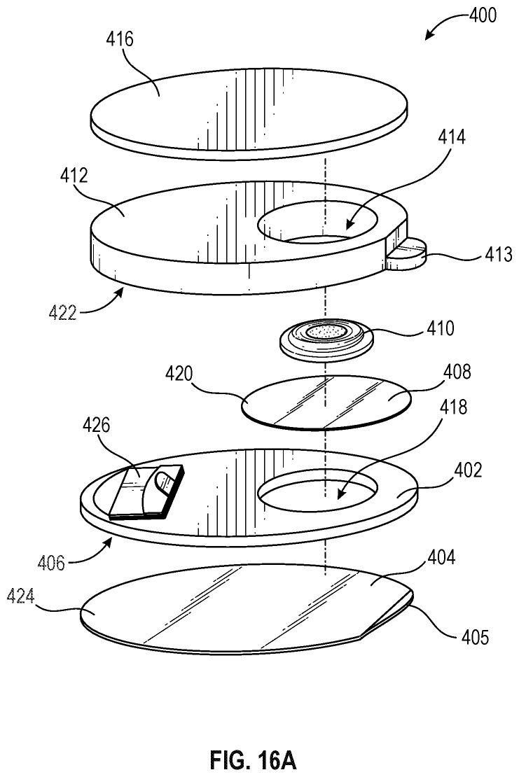

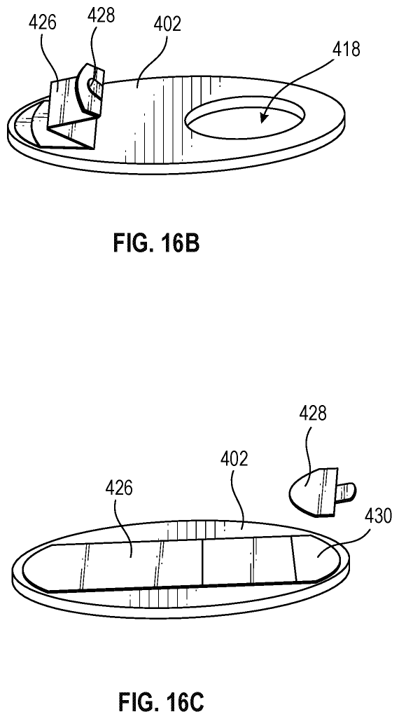

In some embodiments, as shown in FIGS. 16A-C, an adhesive bandage system 400 comprises a first member 402, a release liner 404 (e.g., backing paper) coupled to a first side 406 of the first member 402, and a second member 408 comprising medication or other topical ointment/cream 410. The second member 408 may comprise gauze or other non-stick material that allows for the medication to pass therethrough. A third member 412 may protect a pre-packaged medication 410, as well as while the medication 410 is in use on a patient. For example, the third member 412 may comprise an aperture 414 in the center thereof and have a height greater than the height of the medication 410 on the second member 408, thereby allowing the medication 410 to reside within the aperture 414 (a medication reservoir) during packaging and storage, reducing or preventing the medication 410 from coming into contact with a cover 416. The third member 412 may comprise foam or other materials, allowing it to flex, allowing for the adhesive bandage 400 to be used on different curves of a patient's body. The third member 412 is manufactured from non-absorbent materials so that the medication is not absorbed thereinto. Lastly, a film or cover 416 encloses and protects the medication 410 during storage and while in use.

The second member 408 is received within, or placed over, a first aperture 418 of the first member 402. Because the second member 408 is made from gauze, mesh, or other material allowing the medication 410 to pass or seep therethrough, the medication 410 will contact a patient through the aperture 418 once the release liner 404 is removed. The second member 408 is adhered (preferably non-releasably), around an outer edge 420, to the third member 412. The underside 422 of third member 412 is releasably adhered to top side 424 of the first member 402, allowing a user to remove the third member 412 by peeling it from the first member 402. The third member 412 may further comprise a tab 413 to allow a user to more easily peel it from the first member 402. Additionally, the release liner 404 may comprise a tab 405 to likewise allow for easier gripping and removal from the first side 406 of the first member 402. Further, a wound bandage 426 may be coupled to the first member 402 and configured to cover the aperture 418 once an injection has been administered.

In some embodiments, the wound bandage 426 may be folded, as shown in FIG. 16A so as to not interfere with the aperture 418 and medication 410 as it passes through the aperture 418. In other words, the wound bandage 426 is interposed between the first member 402 and the third member 412, remaining secured therebetween. After a user has peeled or otherwise removed the third member 412 from the first member 402, the bandage 412 is exposed and a first end remains adhered to the first member 402. A user may then unfold and extend the wound bandage 426, as shown in FIGS. 16B and 16C after the injection so as to protect the injection site. In some instances, additional creams, ointments, or other medications may be placed within the aperture 418 and onto the skin before the wound bandage 426 is extended to cover it. The wound bandage 426 may adhere on an opposite end to the first member 412, keeping it secured in place until a user removes the first member 402 from the skin. In other words, a release tab 428 may be removed from the second end 430 of the wound bandage 426, allowing it to be adhered to the first member 402. It will be appreciated that the port/couplers 114 shown in prior embodiments may also be used with this and any other embodiment disclosed herein.

While lidocaine was used as an example above, it will be appreciated that other medications/creams/ointments may be used. Indeed, in some instances, the diameter or other size of the apertures may vary to aid in dosing/uptake depending on the type of medication and the patient (e.g., age, weight, etc.). In some instances, the cover 216, 316, 416 is removably adherable, allowing a user to deposit the desired medication within the aperture 214, 314, 414 of the third member 212, 312, 412 and onto the second member 208, 308, 408.

The bandage adhesive system 100 may be placed onto a finger, the abdomen, thigh, calf, arm, shoulder, or any other regions of exposed skin on the patient. Adhesives typically used in bandages and the like can be used herein.

As a result, the adhesive bandage system 100-400 disclosed herein solves the need for a bandage for use with creams, ointments, and solutions, that allows metered doses, and that provides an indication to a user of the location of the applied medication, assisting in particular with numbing a location before injections or other procedures.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. An adhesive bandage system, comprising:
a first member, comprising:
an aperture, and
an adhesive on a first side;
a release liner removably coupled to the adhesive of the first side of the first member;
a second member at least partially spanning the aperture of the first member and configured to allow medication to pass therethrough;
a third member, comprising:
an adhesive on an underside for removably adhering to a top side of the first member,
an aperture aligned with the second member, the aperture configured to receive a medication therein, contacting the second member; and
a cover adhered to a top side of the third member, the cover sealing the aperture of the third member.

2. The adhesive bandage system of claim 1, wherein the second member comprises a transdermal patch comprising medication.

3. The adhesive bandage system of claim 1, wherein the third member further comprises a tab extending beyond the perimeter of the first member.

4. The adhesive bandage system of claim 1, wherein the first member further comprises a wound bandage coupled to the top side of the first member.

5. The adhesive bandage system of claim 4, wherein when the wound bandage is in a first position, the aperture of the first member is unobstructed, and when the wound bandage is in a second position, the aperture is at least partially covered by the wound bandage.

6. The adhesive bandage system of claim 1, wherein the first member further comprises a coupler configured to receive an intravenous catheter or needle.

7. The adhesive bandage system of claim 1, wherein the medication is lidocaine, benzocaine, testosterone, estrogen, estradiol, ketoprofen, indomethacin, diclofenac, capsaicin, methyl salicylate, topical antibiotics, gabapentin, or topical corticosteroids.

8. The adhesive bandage system of claim 1, wherein the third member further comprises a tab that extends beyond the perimeter of the first member.

9. The adhesive bandage system of claim 1, wherein the cover is removably adhered to the third member.

10. An adhesive bandage system, comprising:
a first member comprising an aperture circumscribed by an adhesive layer on a first side configured to adhere to a patient's skin, and a release liner substantially coextensive with the adhesive layer; and
a second member removably couplable to a second side of the first member, the second member comprising an outer perimeter having an adhesive, the outer perimeter having a diameter greater than a diameter of the aperture of the first member, thereby securing the second member to the second side of the first member and covering the aperture.

11. The adhesive bandage system of claim 10, wherein a medicine reservoir is formed between the release liner and the second member.

12. The adhesive bandage system of claim 11, wherein the medicine reservoir comprises a medication therein.

13. The adhesive bandage system of claim 10, wherein the aperture is configured to receive medication therein.

14. The adhesive bandage system of claim 10, wherein the second member comprises a transdermal patch comprising medication.

15. The adhesive bandage system of claim 10, wherein the aperture is configured to identify an injection site on the patient's skin and to receive a needle therein.

16. The adhesive bandage system of claim 10, wherein the first member further comprises a coupler configured to receive an intravenous catheter or needle.

17. The adhesive bandage system of claim 10, wherein the second member further comprises an occlusive dressing.

18. A method of using an adhesive bandage system for administering medication to a patient, the method comprising:
removing a release liner from an underside of a first member;
adhering a first member of an adhesive bandage to a patient;
administering a topical medication to the patient through an aperture of the first member;
positioning a second member of the adhesive bandage to cover the aperture on the first member; and
covering the second member with a third member, the third member comprising an aperture aligned with at least a portion of the second member; and covering the third member with a cover.

19. The method of claim 18, further comprising:
removing the second member, third member, and cover, from the first member;
using the aperture on the first member to identify an injection site;
inserting a needle through the aperture into the injection site on the patient's skin.

20. The method of claim 19, further comprising extending a wound bandage across at least a portion of the first aperture of the first member.
